(19) United States
(12) Patent Application Publication
(10) Pub. No.: US 2026/0325334 A1
(43) Pub. Date: Oct. 22, 2026

(54) ARM SUPPORT FOR RESTING IN SITTING POSITION

(71) Applicant: NEST Creative Solutions, LLC, Greenwich, CT (US)

(72) Inventors: Samuel A. TAYLOR, Greenwich, CT (US); Nicholas R. ESTIS, Congers, NY (US); Ian D. KOVACEVICH, Carlsbad, CA (US); Kassie BETTS, San Diego, CA (US)

(21) Appl. No.: 19/445,899

(22) Filed: Jan. 12, 2026

Related U.S. Application Data
(60) Provisional application No. 63/743,772, filed on Jan. 10, 2025.

(51) Int. Cl. A61F 5/00

(52) U.S. Cl. CPC A61F 5/00

(57) ABSTRACT

An arm support apparatus includes a tubular component and one or more anchor components and one or more restriction components. The tubular component supports an arm at an elevated position when a person is in a sitting position for resting, including sleeping, while recovering from shoulder surgery. The anchor components fix the tubular component to a bed and keep it from shifting to a different location on the bed during use. The restriction component limits the range of motion of the arm and, in particular, limits backward movement of the elbow of the arm. The arm support apparatus accommodates an arm retained in a sling, brace, cast, or other immobilizing device or restraint. Other contexts of use are also contemplated. In addition to such use for post-op patients, the apparatus can be used for maternity sleep issues, breast feeding, neck support, and sleep apnea, for example.

### CROSS-REFERENCE TO RELATED APPLICATIONS

The present application claims priority under 35 U.S.C. § 119 (e) to, U.S. provisional patent application 63/743,772, filed Jan. 10, 2025, incorporated by reference herein.

### COPYRIGHT STATEMENT

Any new and original work of authorship in this document—including any source code—is subject to copyright protection under the copyright laws of the United States and other countries. Reproduction by anyone of this document as it appears in official governmental records is permitted, but otherwise all other copyright rights whatsoever are reserved.

### INCORPORATION OF COMPUTER PROGRAM LISTING APPENDIX

Submitted concurrently herewith via the USPTO's electronic filing system, and hereby incorporated herein by reference, is a computer program listing appendix. A table setting forth the name and size of files included in the computer program listing appendix is included below.

| File Name | Creation Date | File Size (bytes) |
|---|---|---|
| ASCIFY.txt | Jan. 8, 2026 12:34 | 37,473 |
| readme.txt | Jan. 8, 2026 12:34 | 2,890 |
| install.txt | Jan. 8, 2026 12:34 | 26,082,710 |
| use1.txt | Jan. 8, 2026 12:34 | 14,192,204 |
| use2.txt | Jan. 8, 2026 12:34 | 11,236,510 |

One of these files, “readme.txt”, contains instructions for utilizing “ascify.txt” to convert each other ASCII file in this computer program listing into a compressed “.zip” file. The compressed “.zip” file resulting from “install.txt” comprises a video illustrating the installation of a prototype in accordance with one or more aspects and features of the invention; the compressed “.zip” file resulting from “use l.txt” comprises a video illustrating use of the installed prototype in accordance with one or more aspects and features of the invention; and the compressed “.zip” file resulting from “use2.txt” comprises a video illustrating the further use of the installed prototype in accordance with one or more aspects and features of the invention. Each video file has a “mov” file extension and is compatible, for example, with the Photos app on a Mac and the Media Player app on a PC.

### BACKGROUND OF THE INVENTION

The invention generally relates to apparatus, systems, and methods for supporting an arm while resting and, more particularly, to arm-support apparatus, systems, and methods for improving recovery from shoulder surgery by enhancing the post-operative quality of resting, including sleeping, by a patient.

The greatest complaint from those recovering from shoulder surgery is difficulty in sleeping. Such difficulty can last four-to-eight weeks. This is because most surgeons recommend sling immobilization following an operation during the recovery period, which makes getting high quality rest and sleep difficult. Challenges include unintentional positional changes while sleeping, especially arm movements. Solutions exist for supporting a person while sleeping or otherwise resting, but those are believed to be inadequate. Such solutions include wedge pillows and recliner chairs. Such solutions can be expensive, impractical to transport, and fail to properly restrain or otherwise immobilize movement for proper arm support. Accordingly, it is believed that a need exists for apparatus, systems, and methods that provide a better solution, and that particularly are designed to improve recovery from shoulder surgery by enhancing post-operative sleep quality through proper and comfortable support of an arm, whether such arm is restrained by a sling, brace, or other immobilizing device that may be used when ambulatory. Such a need is believed to be addressed by one or more embodiments in accordance with one or more aspects and features of the present invention.

### SUMMARY OF THE INVENTION

The invention includes many aspects and features. Moreover, while many aspects and features relate to, and are described in, the context of sleep apparatus, systems, and methods for aiding and comforting persons recovering from shoulder surgery, a shoulder injury, or undergoing shoulder therapy, the invention is not limited to use only in such contexts. As will become apparent from the following summaries and detailed descriptions of aspects, features, and one or more embodiments of the invention, the invention has utility outside of such contexts and can be used, for example, by persons without any shoulder condition for support during resting or sleeping. Other contexts include use for maternity sleep issues, breast feeding, neck support, and sleep apnea.

In an aspect of the invention, an arm support apparatus comprises a tubular component and one or more anchor components.

In a feature, an anchor component comprises a fabric sheet.

In a feature, an anchor component comprises a fabric sheet and hook-and-loop fasteners for securing an end edge portion of the fabric sheet to itself in overlapping manner around the tubular component so as to define a passageway for accommodating extension of the tubular component therethrough.

In a feature, an anchor component comprises a fabric sheet and an end edge portion of the fabric sheet that is sewn or otherwise permanently attached to itself so as to define a passageway around the tubular component for accommodating extension of the tubular component therethrough.

In a feature, the tubular component comprises a foam subcomponent and a spine subcomponent, both of which are enclosed within an outer fabric covering of the tubular component. Preferably, the spine subcomponent is rigid but also manually bendable and it enables the tubular component to be manually manipulated into and remain in different configurations; the tubular component is transitionable between a horseshoe configuration, a generally straight configuration, and a spiral configuration; each configuration of the tubular component is stable insofar as the tubular component remains in such configuration until a sufficient force is applied for manually manipulating the tubular component into a different configuration; and such a force preferably is greater than the force arising from the weight of an arm that is supported at an elevation by the tubular component.

In a feature, each anchor component comprises a fabric sheet that has a first end portion that is secured on a portion of the tubular component and that has an opposite end portion that freely drapes or hangs from the tubular component. Preferably, the first end portion of the anchor component is doubled over on itself with an end edge portion being sewn or otherwise attached to itself such that the tubular component is hemmed in by the first end portion; or alternatively, the first end portion of the anchor component surrounds the tubular component and forms a passageway through which the tubular component extends, thereby enabling the anchor component to be secured onto the tubular component without fixing the location at which it is secured and enabling the anchor component to be repositioned along the tubular component by sliding the first end portion along the tubular component. In other possible embodiments, an anchor component may be directly attached to the tubular component, such as by hook-and-loop fasteners or by being sewn to the outer component of the tubular component.

In a feature, the arm support apparatus comprises a pocket at each of its opposite distal ends. The pocket is configured for holding a mobile phone or remote-control device for a television or other audiovisual equipment. Preferably, the pocket is formed by overlapping fabric sections of the tubular component and, in particular, the pocket is formed in an outer fabric cover of the tubular component.

In a feature, the tubular component comprises an outer fabric covering, and a zipper fastening mechanism for providing access to an interior of the tubular component.

In a feature, the tubular component comprises a foam subcomponent. Preferably, the foam subcomponent defines an interior passageway located along a longitudinal core thereof; two end cap subcomponents cap off the interior passageway of the foam subcomponent; and a spine subcomponent is located within the interior passageway between the end cap subcomponents. The spine subcomponent may comprise a modular tube or a hose, such as those sold under the loc-line® brand. Preferably, the end cap subcomponents are adhered, welded, or otherwise attached to opposite distal ends of the foam subcomponent; and the spine subcomponent comprises a modular tube or hose. In another feature, the arm support apparatus further comprises a support component for supporting one or more legs of a person as a leg rest or ottoman when sitting. The support component preferably comprises a first foam subcomponent and a second foam subcomponent forming a composite cushion subcomponent. The support component further may comprise a sheet subcomponent attached at a longitudinal end to the composite cushion subcomponent and extending away to an opposite longitudinal end that is unattached and is configured to be positioned so as to anchor the composite cushion component during use.

In another aspect, a tubular component comprises a foam subcomponent having an outer fabric covering and a spine subcomponent extending along a longitudinal axis of the foam subcomponent and defining an interior passageway, wherein the outer fabric covering does not cover a longitudinal end of the tubular component such that an end of the interior spine subcomponent is exposed and the interior passageway of the spine subcomponent is made accessible, and wherein an end cap subcomponent is inserted into the interior passageway of the end of the spine subcomponent.

In a feature, the end cap subcomponent forms an outermost end of the tubular component.

In a feature, the end cap subcomponent has a threaded opening on one side and a plug portion on the opposite side, the plug portion fitting in friction fit within the interior passageway of the spine subcomponent at the exposed end of the interior spine subcomponent. The threaded opening of the end cap subcomponent preferably is configured to receive in threaded engagement a threaded plug portion of another quick connect subcomponent, the quick connect subcomponent having an opposite threaded plug portion.

In another aspect, a tubular component comprises multiple tubular components join together in end-to-end relation, each of the multiple tubular components comprising a foam subcomponent having an outer fabric covering and a spine subcomponent extending along a longitudinal axis of the foam subcomponent and defining an interior passageway, wherein the outer fabric covering does not cover a longitudinal end of the tubular component such that an end of the interior spine subcomponent is exposed and the interior passageway of the spine subcomponent is made accessible.

In another aspect, a tubular component comprises a foam subcomponent having an outer fabric covering and a spine subcomponent extending along a longitudinal axis of the foam subcomponent and defining an interior passageway. The outer fabric covering does not cover a longitudinal end of the tubular component such that an end of the interior spine subcomponent is exposed and the interior passageway of the spine subcomponent is made accessible, and a wireless speaker subcomponent is inserted into the interior passageway of the end of the spine subcomponent, the wireless speaker subcomponent comprising a plug portion for insertion in friction fit into the interior passageway of the spine subcomponent of the tubular component.

In a feature, the wireless speaker subcomponent is configured to connect to an electronic device for playing music.

In a feature, the wireless speaker subcomponent further comprises an internal, rechargeable power source and a power button for turning on and off the wireless speaker subcomponent while inserted in the interior passageway of the spine subcomponent of the tubular component.

In another aspect, a tubular component comprises a foam subcomponent having an outer fabric covering and a spine subcomponent extending along a longitudinal axis of the foam subcomponent and defining an interior passageway, the outer fabric covering does not cover a longitudinal end of the tubular component such that an end of the interior spine subcomponent is exposed and the interior passageway of the spine subcomponent is made accessible, and an air circulation subcomponent comprising a fan is inserted into the interior passageway of the end of the spine subcomponent, the air circulation subcomponent comprising a plug portion for insertion in friction fit into the interior passageway of the spine subcomponent of the tubular component, the air circulation subcomponent being configured to cause air to flow from exterior of the tubular component into and through the interior passageway of the spine subcomponent.

In another aspect, the air circulation subcomponent further comprises an internal heating mechanism, an internal cooling mechanism, or both for heating/cooling air flowing through the air circulation subcomponent.

In another aspect, the spine subcomponent is ventilated.

In another aspect, the tubular component further comprises ventilation passageways extending through the fabric cover, the foam subcomponent, and the spine subcomponent for facilitating air flow from the spine subcomponent out of the tubular component.

In a feature, the ventilation passageways form an array of one or more rows of exterior openings for airflow from the interior passageway of the spine subcomponent out of the tubular component.

In a feature, the tubular component further comprises a wireless speaker subcomponent inserted into the interior passageway of the opposite longitudinal end of the spine subcomponent, the wireless speaker subcomponent comprising a plug portion for insertion in friction fit into the interior passageway of the spine subcomponent of the tubular component. Preferably, the wireless speaker subcomponent caps the end of the interior passageway of the spine subcomponent and airflow longitudinally therethrough.

In another aspect, a method of making a foam subcomponent of a tubular component for an arm support apparatus comprises transforming a sheet of foam into a tube and fixing the sheet of foam in the configuration of the tube.

In a feature, the foam is memory foam.

In a feature, the sheet of foam is transformed by rolling. Preferably, the sheet of foam is fixed in the configuration of the tube by welding. Alternatively, the sheet of foam preferably is fixed in the configuration of the tube using an adhesive.

In another aspect, an arm support apparatus comprises a tubular component and one or more restriction components that are attachable to the tubular component and designed to limit the movement of the arm, and preferably the elbow, when used.

In a feature, the restriction component comprises a block and a strap.

Preferably, a distal end of the strap is permanently attached to an end of the block, and the other distal end of the strap is looped through a D-ring that is attached to another end of the block, whereby the strap extends back over itself to fasten proximate the first distal end; and hook-and-loop fasteners on the strap for detachably securing the second distal end to the strap proximate the first distal end after positioning of the strap around the tubular component for securing the restriction component to the tubular component. Additionally, the block preferably comprises an outer fabric covering, foam subcomponent enclosed within the outer fabric covering, and a zipper fastening mechanism providing access to the interior of the block and the enclosed foam subcomponent. Preferably, the foam subcomponent comprises a memory foam and defines and gives shape to the block.

In another aspect, a method of providing an arm support apparatus comprises disposing a plurality of separate components for assembly by a user in a container, the separate components comprising a tubular component, one or more anchor components, and one or more restriction components. Preferably, two anchor component and two restriction components are provided with the tubular component.

In a feature, the tubular component is configured to wrap around and contain the other components when packaged in a container such as a box. Preferably, the tubular component is in a spiral configuration in wrapping around and containing the other components.

Another aspect relates to a method of installing an arm support apparatus on a bed. The method comprises positioning a tubular component along one or more edges of the bed and tucking an end portion of one or more anchor components between a mattress and a box spring of the bed.

In a feature, the arm support apparatus is positioned at a corner of the bed.

In a feature, the arm support apparatus includes two anchor members each located on a different side of the bed proximate a corner of the bed, whereby the anchor components retain the arm support apparatus to the bed and keep portions of the tubular component extending through the passageway and between the anchor components from moving away from the edges of the bed during use.

In a feature, the arm support apparatus includes a “free” end portion that is bendable and transitionable relative to a person sitting on the bed at the corner of the bed so as to support an inside arm of the person during use of the arm support apparatus. Preferably, the tubular component preferably is between about five feet and about ten feet in longitudinal length between its opposite distal ends, with the free end portion being between about two feet and about three feet, or more, in length; the free end portion extends about 25% to about 40%, or more, of the total longitudinal length of the tubular component; or about 60% to about 75% of the tubular component is secured by the anchor components along one or more edges of the bed when installed, with the remaining length of the tubular component being free to extend from the edge and manipulated into a desired configuration for support of an arm.

In a feature, the method includes wrapping the anchor component about a portion of the tubular component and securing the anchor component to itself in a detachable manner.

In a feature, the method includes wrapping the anchor component about a portion of the tubular component and securing the anchor component to itself using hook-and-loop fasteners.

In a feature, the method includes positioning one or more pillows at a bend in the tubular component for supporting a person's back in a sitting position for supporting a person at an incline of between 30 to 90 degrees when in the sitting position for resting, including sleeping. Preferably, the incline is between 35 and 45 degrees for comfort.

In a feature, the method further comprises supporting an arm at an elevation above a person's thigh using the free end of the tubular component. Preferably, the method further restricts backward movement of the arm's elbow using a restriction component that is removably attached to the tubular component. The arm may further be retained within a sling, brace, or other immobilizing device.

Another aspect comprises a method of using a tubular component as disclosed herein for sleeping.

Another aspect comprises a method of using a tubular component as disclosed herein for resting in an office chair.

Another aspect comprises a method of using a tubular component as disclosed herein for attending to a child.

Another aspect comprises a method of using a tubular component as disclosed herein as a neck pillow when traveling.

Additional aspects and features of the invention are disclosed in the appendices and in the videos of the computer program listing, incorporated herein by reference.

In addition to the aforementioned aspects and features of the invention, it should be noted that the invention further encompasses the various logical combinations and subcombinations of such aspects and features. Thus, for example, claims in this or a divisional or continuing patent application or applications may be separately directed to any aspect, feature, or embodiment disclosed herein, or combination thereof, without requiring any other aspect, feature, or embodiment.

### BRIEF DESCRIPTION OF THE DRAWINGS

One or more preferred embodiments of the invention now will be described in detail with reference to the accompanying drawings, wherein the same elements are referred to with the same reference numerals.

FIG. 1 is an orthogonal view of an embodiment of an arm support apparatus in accordance with one or more aspects and features of the invention.

FIG. 2 is an orthogonal view of a portion of the arm support apparatus of FIG. 1 that has been installed on a mattress.

FIG. 3 is an orthogonal view of a portion of another arm support apparatus in accordance with one or more aspects and features of the invention that has been installed on a mattress.

FIG. 4 is an orthogonal view of a top of an embodiment of a tubular component of an arm support apparatus in accordance with one or more aspects and features of the invention, wherein the tubular component is shown in a horseshoe configuration.

FIG. 5 is an orthogonal view of a bottom of the tubular component of FIG. 4, wherein the tubular component is shown in a straight configuration.

FIG. 6 is an orthogonal view of the tubular component of FIG. 4 after having been manipulated into a generally straight orientation.

FIG. 7 is an orthogonal view of an end of the tubular component of FIG. 6 showing a pocket of the tubular component in an opened configuration.

FIG. 8 is a perspective view of an embodiment of a tubular component of an arm support apparatus in accordance with one or more aspects and features of the invention, which view shows a front and side of the tubular component.

FIG. 9 is a perspective view of the tubular component of FIG. 8 that shows a rear and side of the tubular component.

FIG. 10 illustrates steps in the process of making a tubular component of an arm support apparatus in accordance with one or more aspects and features of the invention.

FIG. 11 is an orthogonal view of a foam subcomponent of a tubular component of an arm support apparatus in accordance with one or more aspects and features of the invention.

FIG. 12 is an orthogonal view of a cross-section of the foam subcomponent of FIG. 11 viewed along the line 12-12 in FIG. 11.

FIG. 13 is an orthogonal view of an end cap subcomponent of a tubular component of an arm support apparatus in accordance with one or more aspects and features of the invention.

FIG. 14 illustrates subsequent steps in the process of making the tubular component after the steps illustrated in FIG. 10.

FIG. 15 is an orthogonal view of a spine subcomponent of a tubular component of an arm support apparatus in accordance with one or more aspects and features of the invention.

FIG. 16 is an orthogonal view of an end of the spine subcomponent of FIG. 15 after being capped with an end cap subcomponent.

FIG. 17 is a cross-sectional view of a tubular subcomponent assembly of a tubular component of an arm support apparatus in accordance with one or more aspects and features of the invention.

FIG. 18 is an orthogonal view of part of a spine subcomponent in accordance with one or more aspects and features of the invention.

FIG. 19 is an elevational view of part of the spine subcomponent of FIG. 18.

FIG. 20 is an orthogonal view of an anchor component of an arm support apparatus in accordance with one or more aspects and features of the invention.

FIG. 21 is an orthogonal view of another anchor component in accordance with one or more aspects and features of the invention.

FIG. 22 is a perspective view of a side of an anchor component of an arm support apparatus in accordance with one or more aspects and features of the invention.

FIG. 23 is a perspective view of a side of another anchor component in accordance with one or more aspects and features of the invention.

FIG. 24 is an orthogonal view of a bottom of a restriction component of an arm support apparatus in accordance with one or more aspects and features of the invention.

FIG. 25 is an orthogonal view of a front of the restriction component of FIG. 24.

FIG. 26 is an orthogonal view of a foam subcomponent of the restriction component of FIG. 24.

FIG. 27 is a perspective view of a front of a restriction component of an arm support apparatus in accordance with one or more aspects and features of the invention.

FIG. 28 is another perspective view of the front of the restriction component of FIG. 27.

FIG. 29 is a perspective view of an embodiment of an arm support apparatus in accordance with one or more aspects and features of the invention.

FIG. 30 is a perspective view of the arm support apparatus of FIG. 29.

FIG. 31 is another perspective view of the arm support apparatus of FIG. 29.

FIG. 32 is a perspective, close-up view of a portion of the arm support apparatus of FIG. 29.

FIG. 33 is a perspective, close-up view of another portion of the arm support apparatus of FIG. 29.

FIG. 34 is a perspective view of an embodiment of an arm support apparatus installed and ready for use in accordance with one or more aspects and features of the invention.

FIG. 35 is a perspective view of the arm support apparatus of FIG. 34 in use in accordance with one or more aspects and features of the invention.

FIG. 36 is another perspective view of the arm support apparatus of FIG. 34 in use in accordance with one or more aspects and features of the invention.

FIG. 37 is a perspective view of five separate components that form a preferred arm support apparatus for commercialization in accordance with one or more aspects and features of the invention.

FIG. 38 is a perspective view of the five separate components of FIG. 37 being bundled and placed within a container for storage or shipment for commercialization in accordance with one or more aspects and features of the invention.

FIG. 39 is an illustration of a use of an embodiment of a tubular component in accordance with one or more aspects and features of the invention.

FIG. 40 is an illustration of a use of embodiments of a tubular component and a restriction component in accordance with one or more aspects and features of the invention.

FIG. 41 is an illustration of a use of embodiments of a tubular component and a restriction component in accordance with one or more aspects and features of the invention.

FIG. 42 is an illustration of a use of another embodiment of a tubular component in accordance with one or more aspects and features of the invention.

FIG. 43 is a perspective view of an end of an embodiment of a tubular component in accordance with one or more aspects and features of the invention.

FIG. 44 is an orthogonal view of an end cap subcomponent for use with the tubular component of FIG. 43.

FIG. 45 is an orthogonal view of a quick connect subcomponent for use in connecting tubular components of which FIG. 43 is representative.

FIG. 46 is an orthogonal view of a wireless speaker subcomponent for use with a tubular component of which FIG. 43 is representative.

FIG. 47 is an orthogonal view of an air circulation subcomponent having a fan for use with a tubular component of which FIG. 43 is representative.

FIG. 48 is a perspective view of an end of an embodiment of a tubular component with which the air circulation subcomponent of FIG. 47 is used in accordance with one or more aspects and features of the invention.

FIG. 49 is a perspective, exploded view of embodiments of an air circulation subcomponent and a wireless speaker subcomponent of a ventilated tubular component in accordance with one or more aspects and features of the invention.

FIG. 50 is an elevational view of the air circulation subcomponent and wireless speaker subcomponent of a tubular component of FIG. 50 wherein a portion of the tubular component is shown in cross-section to reveal a ventilated spine subcomponent therein.

FIG. 51 is an illustration of a use of the heating/cooling component and wireless speaker component of a tubular component of FIG. 50.

FIG. 52 is an elevational, orthogonal view of an optional component for use with an arm support apparatus in accordance with one or more aspects and features of the invention.

FIG. 53 is a top plan, orthogonal view of the component of FIG. 52.

FIG. 54 is another orthogonal view component of FIG. 52.

FIG. 55 is a perspective view of another optional component for use with an arm support apparatus in accordance with one or more aspects and features of the invention.

### DETAILED DESCRIPTION

As a preliminary matter, it will readily be understood by one having ordinary skill in the relevant art (“Ordinary Artisan”) that the invention has broad utility and application. Furthermore, any embodiment discussed and identified as being “preferred” is considered to be part of a best mode contemplated for carrying out the invention. Other embodiments also may be discussed for additional illustrative purposes in providing a full and enabling disclosure of the invention. Furthermore, an embodiment of the invention may incorporate only one or a plurality of the aspects of the invention disclosed herein; only one or a plurality of the features disclosed herein; or combination thereof. As such, many embodiments are implicitly disclosed herein and fall within the scope of what is regarded as the invention.

Accordingly, while the invention is described herein in detail in relation to one or more embodiments, it is to be understood that this disclosure is illustrative and exemplary of the invention and is made merely for the purposes of providing a full and enabling disclosure of the invention. The detailed disclosure herein of one or more embodiments is not intended, nor is to be construed, to limit the scope of patent protection afforded the invention in any claim of a patent issuing here from, which scope is to be defined by the claims and the equivalents thereof. It is not intended that the scope of patent protection of the invention be defined by reading into any claim a limitation found herein that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the invention. Accordingly, it is intended that the scope of patent protection afforded the invention be defined by the issued claim(s) rather than the description set forth herein.

Additionally, it is important to note that each term used herein refers to that which the Ordinary Artisan would understand such term to mean based on the contextual use of such term herein. To the extent that the meaning of a term used herein—as understood by the Ordinary Artisan based on the contextual use of such term-differs in any way from any particular dictionary definition of such term, it is intended that the meaning of the term as understood by the Ordinary Artisan should prevail.

With regard solely to construction of any claim with respect to the United States, no claim element is to be interpreted under 35 U.S.C. 112 (f) unless the explicit phrase “means for” or “step for” is actually used in such claim element, whereupon this statutory provision is intended to and should apply in the interpretation of such claim element. With regard to any method claim including a condition precedent step, such method requires the condition precedent to be met and the step to be performed at least once but not necessarily every time during performance of the claimed method.

Furthermore, it is important to note that, as used herein, “comprising” is open-ended insofar as that which follows such term is not exclusive. Additionally, “a” and “an” each generally denotes “at least one” but does not exclude a plurality unless the contextual use dictates otherwise. Thus, reference to “a picnic basket having an apple” is the same as “a picnic basket comprising an apple” and “a picnic basket including an apple”, each of which identically describes “a picnic basket having at least one apple” as well as “a picnic basket having apples”; the picnic basket further may contain one or more other items beside an apple. In contrast, reference to “a picnic basket having a single apple” describes “a picnic basket having only one apple”; the picnic basket further may contain one or more other items beside an apple. In contrast, “a picnic basket consisting of an apple” has only a single item contained therein, i.e., one apple; the picnic basket contains no other item.

When used herein to join a list of items, “or” denotes “at least one of the items” but does not exclude a plurality of items of the list. Thus, reference to “a picnic basket having cheese or crackers” describes “a picnic basket having cheese without crackers”, “a picnic basket having crackers without cheese”, and “a picnic basket having both cheese and crackers”; the picnic basket further may contain one or more other items beside cheese and crackers.

When used herein to join a list of items, “and” denotes “all of the items of the list”. Thus, reference to “a picnic basket having cheese and crackers” describes “a picnic basket having cheese, wherein the picnic basket further has crackers”, as well as describes “a picnic basket having crackers, wherein the picnic basket further has cheese”; the picnic basket further may contain one or more other items beside cheese and crackers.

The phrase “at least one” followed by a list of items joined by “and” denotes an item of the list but does not require every item of the list. Thus, “at least one of an apple and an orange” encompasses the following mutually exclusive scenarios: there is an apple but no orange; there is an orange but no apple; and there is both an apple and an orange. In these scenarios if there is an apple, there may be more than one apple, and if there is an orange, there may be more than one orange. Moreover, the phrase “one or more” followed by a list of items joined by “and” is the equivalent of “at least one” followed by the list of items joined by “and”.

Referring now to the drawings, one or more preferred embodiments of the invention are next described. The following description of one or more preferred embodiments is merely exemplary in nature and is in no way intended to limit the invention, its implementations, or uses.

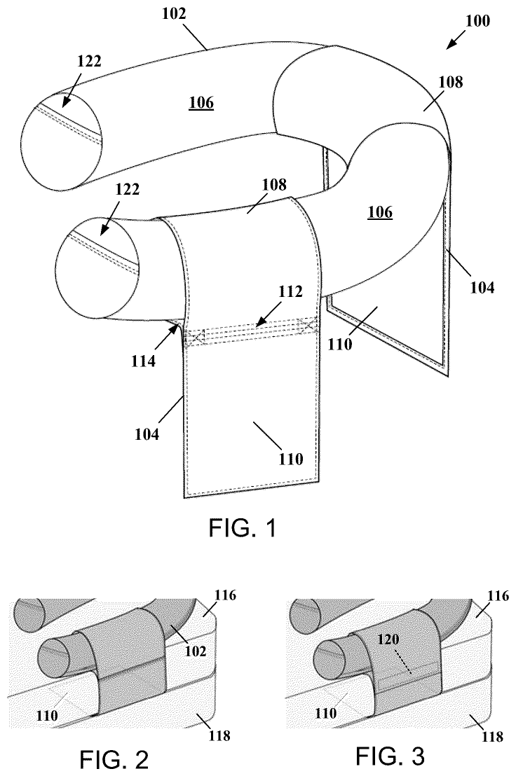

FIG. 1 is an orthogonal view of an embodiment of an arm support apparatus 100 in accordance with one or more aspects and features of the invention. The arm support apparatus comprises a tubular component 102 and one or more anchor components 104, two of which are shown as being part of the arm support apparatus 100 in FIG. 1.

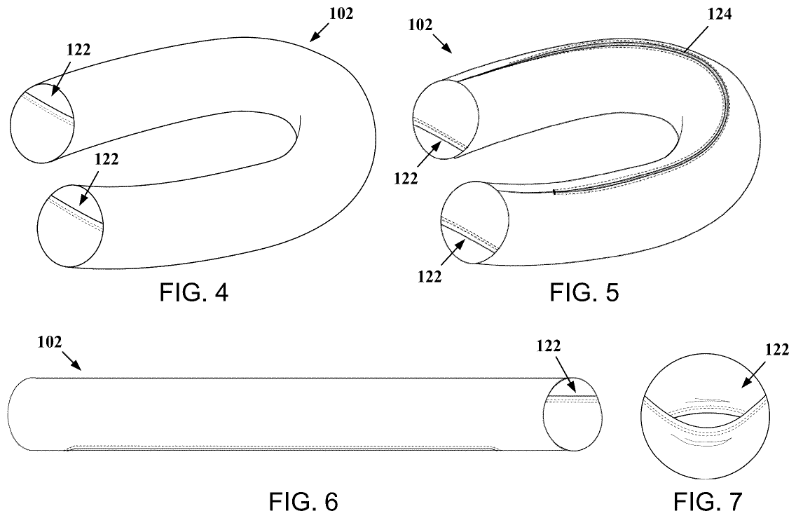

With regard to the tubular component 102, FIG. 4 is an orthogonal view of a top of the tubular component 102; and FIG. 5 is an orthogonal view of a bottom of the tubular component 102. The tubular component 102 comprises a foam subcomponent 314 (see FIG. 14) and a spine subcomponent 314 (see FIG. 15), both of which are enclosed within an outer fabric covering 106 of the tubular component 102. The spine subcomponent 124 is rigid but also manually bendable and it enables the tubular component 102 to be manually manipulated into and remain in different configurations. For example, as shown in FIG. 1, the tubular component 102 is in a horseshoe configuration. In contrast, FIG. 6 is an orthogonal view of the tubular component 102 after having been manipulated into a generally straight configuration from the horseshoe configuration shown in FIGS. 1, 4, and 5.

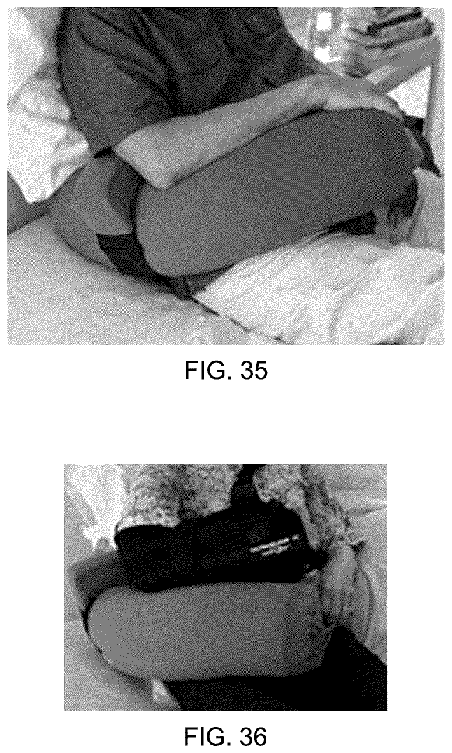

Each configuration of the tubular component 102 is stable insofar as the tubular component 102 will remain in such configuration until a sufficient force is applied for manually manipulating the tubular component 102 into a different configuration. Such a force preferably is greater than the force arising from the weight of an arm that is supported at an elevation by the tubular component 102 in accordance with the intended use of the arm support apparatus 102. Such use is shown, for example, in FIGS. 35 and 36 and is further disclosed in the videos of the computer program listing, incorporated herein by reference.

Each anchor component 104 comprises a fabric sheet that has a first end portion 108 that is secured on a portion of the tubular component 102 and that has an opposite end portion 110 that freely drapes or hangs from the tubular component 102. Furthermore, FIGS. 20-21 perhaps best illustrate the anchor component 104.

The first end portion 108 of the anchor component 104 is doubled over on itself with an end edge portion being sewn or otherwise attached to itself at 112 such that the tubular component 102 is hemmed in by the first end portion 108; the first end portion 108 of the anchor component 104 surrounds the tubular component 102 and forms a passageway 114 through which the tubular component 102 extends. This enables the anchor component 104 to be secured onto the tubular component 102 without fixing the location at which it is secured. The anchor component 104 thus can be repositioned along the tubular component 102 by sliding the first end portion 108 along the tubular component 102.

FIG. 2 is an orthogonal view of a portion of the arm support apparatus 100 of FIG. 1 that has been installed on a bed. The arm support apparatus 100 is installed on the bed by positioning the tubular component 102 along one or more edges of the bed and tucking the end portion 110 of each anchor component 104 between a mattress 116 and a box spring 118 of the bed.

As shown in FIG. 2, the arm support apparatus 100 is positioned at a corner of the bed. Preferably the arm support apparatus 100 includes two anchor members 104 each located on a different side of the bed proximate the corner, whereby the anchor components 104 retain the arm support apparatus 100 to the bed and keep portions of the tubular component 102 extending through the passageway 114 and between the anchor components 104 from moving away from the edges of the bed during use. In this respect, the arm support apparatus 100 preferably includes a “free” end portion that is bendable and transitionable relative to a person sitting on the bed at the corner of the bed so as to support an inside arm of the person (the arm furthest from an edge of the bed) during use of the arm support apparatus 100.

To facilitate this, the tubular component 102 preferably is between about five feet and about ten feet in longitudinal length between its opposite distal ends, with the free end portion being between about two feet and about three feet, or more, in length. Described in another way, preferably the free end portion extends about 25% to about 40%, or more, of the total longitudinal length of the tubular component 102. Conversely, preferably about 60% to about 75% of the tubular component 102 is secured by the anchor components 104 along one or more edges of the bed when installed, with the remaining length of the tubular component 102 being free to extend from the edge and manipulated into a desired configuration for support of an arm.

FIG. 3 is an orthogonal view of a portion of another arm support apparatus in accordance with one or more aspects and features of the invention that has been installed on a mattress. The arm support apparatus of FIG. 3 is similar to that of FIG. 2 and differs in that the anchor component is wrapped about a portion of the tubular component and secured to itself not by sewing but instead is secured in a detachable manner and preferably as shown, by hook-and-loop fasteners 120.

Returning again to FIG. 1, the arm support apparatus 100 preferably includes a pocket 122 at each of its opposite distal ends. The pocket preferably is configured for holding a mobile phone or remote-control device for a television or other audiovisual equipment. The pocket 122 provides a convenient location for temporarily keeping a phone or remote-control device while the support arm apparatus 100 is being used by a person. The pocket 122 is formed by overlapping fabric sections of the tubular component 102 and the pocket 122 is shown in an opened configuration in FIG. 7.

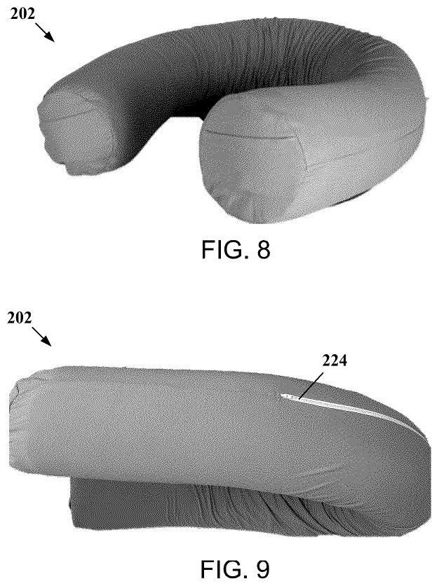

FIG. 8 is a perspective view of an embodiment of a tubular component 202 of an arm support apparatus in accordance with one or more aspects and features of the invention, which view shows a front and side of the tubular component. The tubular component 202 is a prototype that has been reduced to practice and that corresponds to the tubular component 102. FIG. 9 is a perspective view of the tubular component 202 that shows a rear and side of the tubular component 202. A zipper fastening fastener 224 is provided in the outer fabric covering of the tubular component 102 as shown in FIG. 9 and corresponds to a zipper fastening mechanism 124 of the tubular component 102 shown in FIG. 5. The zipper fastening mechanism provides access to an interior of the tubular component and enables the outer fabric covering to be removed and washed, when needed.

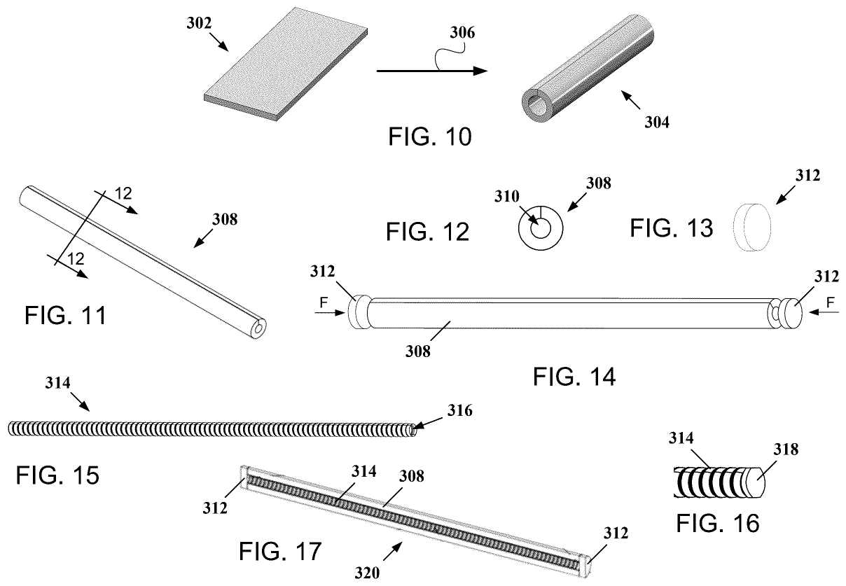

FIG. 10 illustrates steps in the process of making a tubular component of an arm support apparatus in accordance with one or more aspects and features of the invention. In particular, this figure illustrates a step of transforming a sheet 302 of foam into a tube 304—such as by rolling 306 the sheet 302—and welding or otherwise fixing the sheet 302 of foam in the configuration of the tube 304. The foam preferably is a memory foam.

FIG. 11 is an orthogonal view of a foam subcomponent 308 of a tubular component of an arm support apparatus in accordance with one or more aspects and features of the invention. The foam subcomponent 308 comprises the tube 304 of FIG. 10.

FIG. 12 is an orthogonal view of a cross-section of the foam subcomponent 304 taken along the line 12-12 in FIG. 11. The foam subcomponent 308 defines an interior passageway 310 located along its longitudinal core.

FIG. 13 is an orthogonal view of an end cap subcomponent 312 of a tubular component of an arm support apparatus in accordance with one or more aspects and features of the invention. Preferably, two such end cap subcomponents 312 are used to cap off the interior passageway 310 of the foam subcomponent 308 by adhering, welding, or otherwise attaching the end cap subcomponents 312 to opposite distal ends of the foam subcomponent 308, which step is illustrated by arrows F in FIG. 14. As such, FIG. 14 illustrates another step in the process of making the tubular component 102 after the steps illustrated in FIG. 10.

FIG. 15 is an orthogonal view of a spine subcomponent 314 of a tubular component of an arm support apparatus in accordance with one or more aspects and features of the invention. The spine subcomponent 314 includes a hollow interior passageway which preferably is capped at its opposite distal ends by an end cap subcomponent 318, as shown in FIG. 16.

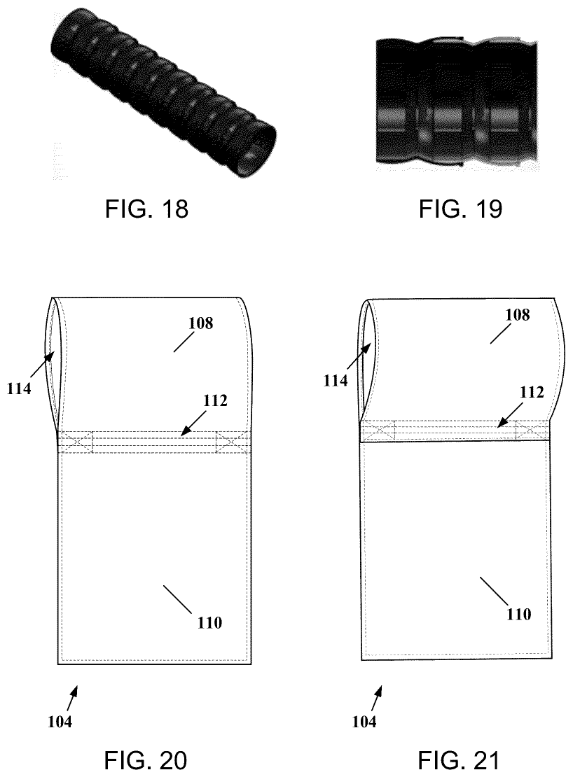

The spine subcomponent preferably comprises a modular tube or hose that is sold under the brand loc-line® by Lockwood Products Inc. of Lake Oswego, Oregon, although other modular tubes and hoses may be used in embodiments in accordance with aspects and features of the invention. FIGS. 18 and 19 illustrate such a preferred tube. FIG. 18 is an orthogonal view of part of such a spine subcomponent; and FIG. 19 is an elevational view of part of the spine subcomponent of FIG. 18. In any case, the modular hose or tube preferably comprises an elongate inner core and is adjustable into multiple different configurations. The elongate tube may be made up of multiple interconnected modular hollow polymeric pieces that are rotatably coupled together to allow the tube to be adjustable while also retaining its shape and/or configuration.

FIG. 17 is a cross-sectional view of the spine subcomponent 314 after being capped with an end cap subcomponent 312 at each distal end and disposed within the passageway 310 of the foam subcomponent 308, with end cap subcomponents 312 thereafter having been attached to the distal ends of the foam subcomponent 308. A resulting tubular subcomponent assembly 320 is shown in FIG. 17. Enclosure of the tubular subcomponent assembly 320 within the fabric covering then forms a preferred tubular component of an arm support apparatus.

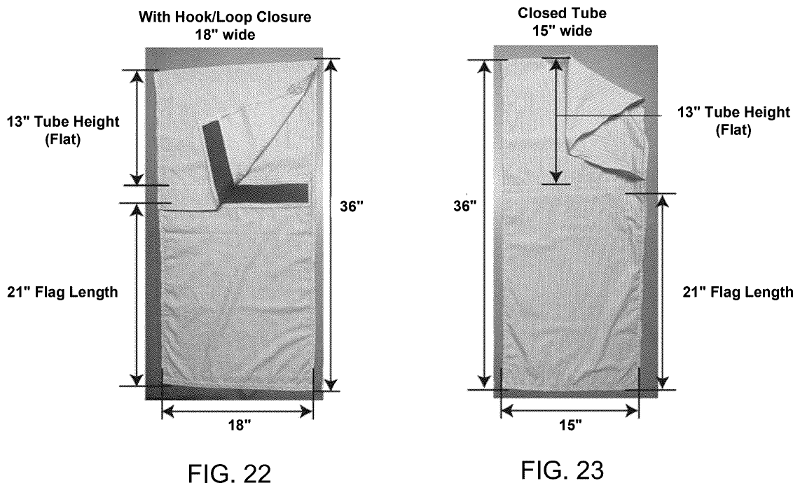

FIG. 22 is a perspective view of a side of an anchor component of an arm support apparatus that has been reduced to practice in accordance with one or more aspects and features of the invention. The anchor component of FIG. 22 includes hook-and-loop fasteners for securing the end edge portion of the fabric sheet to itself. In contrast, FIG. 23 is a perspective view of the opposite side of a side of an anchor component of an arm support apparatus that has been reduced to practice in accordance with one or more aspects and features of the invention, wherein the anchor component of FIG. 23 has been sewn or otherwise permanently attached to itself. Preferred dimensions of the anchor components are illustrated in FIGS. 22 and 23.

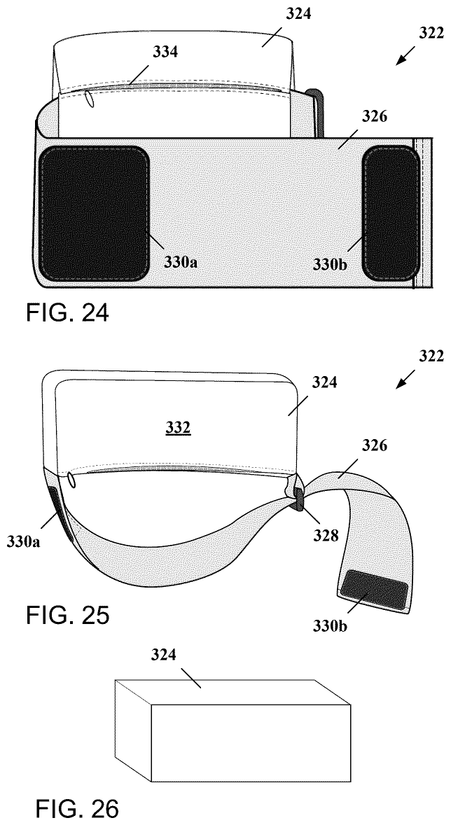

Some preferred embodiments of arm support apparatus include one or more restriction components that are attachable to the tubular component and designed to limit the movement of the arm, and preferably the elbow, when used. FIG. 24 is an orthogonal view of a bottom of such a restriction component 322 of an arm support apparatus in accordance with one or more aspects and features of the invention. FIG. 25 is an orthogonal view of a front of the restriction component 322 of FIG. 24.

The restriction component 322 preferably comprises a block 324 and a strap 326. A distal end of the strap 326 is permanently attached to an end of the block 324, and the other distal end of the strap is looped through a D-ring 328 for extending back over itself to fasten proximate the first distal end. Preferably, hook-and-loop fasteners 330a,330b are adhered, sewn, welded, or otherwise permanently attached to the strap for detachably securing the second distal end to the strap proximate the first distal end after positioning of the strap around the tubular component.

The block 324 comprises an outer fabric covering 332 and a zipper fastening mechanism 334. The zipper fastening mechanism 334 provides access to an interior of the block 324 and enables the outer fabric covering 322 to be removed and washed, when needed. A foam subcomponent 336 preferably comprising memory foam is enclosed within the outer fabric covering 322 and defines and gives shape to the block 324. FIG. 26 is an orthogonal view of such a foam subcomponent 336 of the restriction component 322.

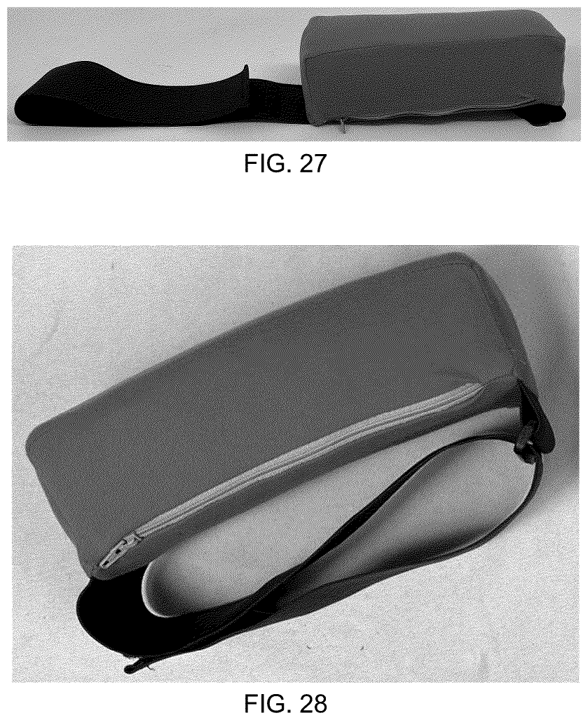

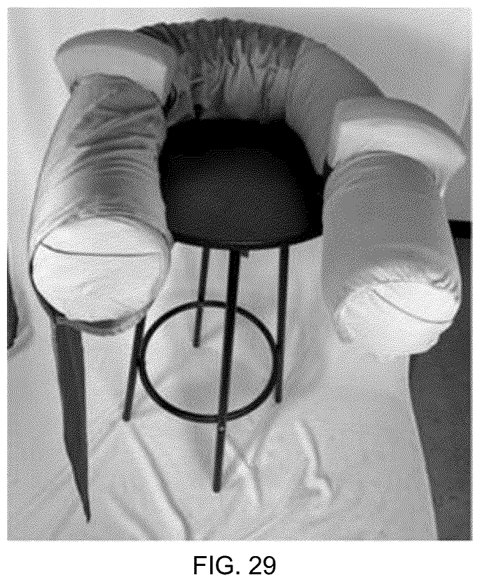

FIG. 27 is a perspective view of a front of a restriction component of a prototype arm support apparatus that has been reduced to practice for use in accordance with one or more aspects and features of the invention. FIG. 28 is another perspective view of the front of the restriction component of FIG. 27.

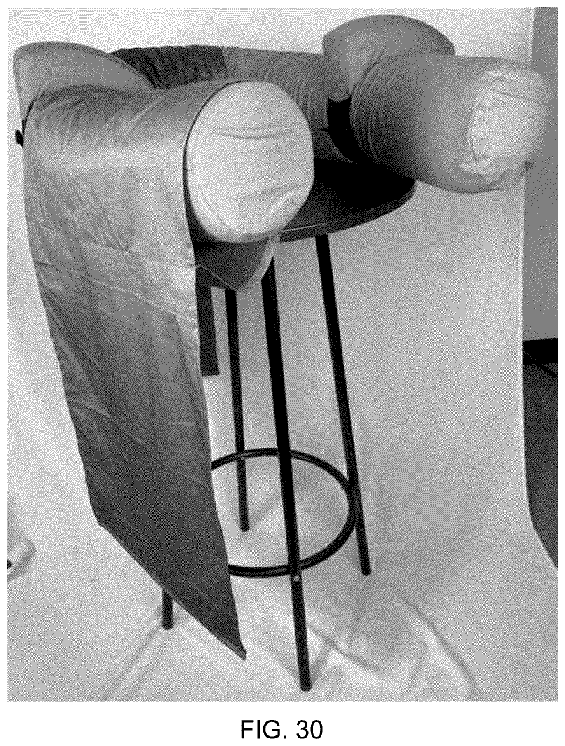

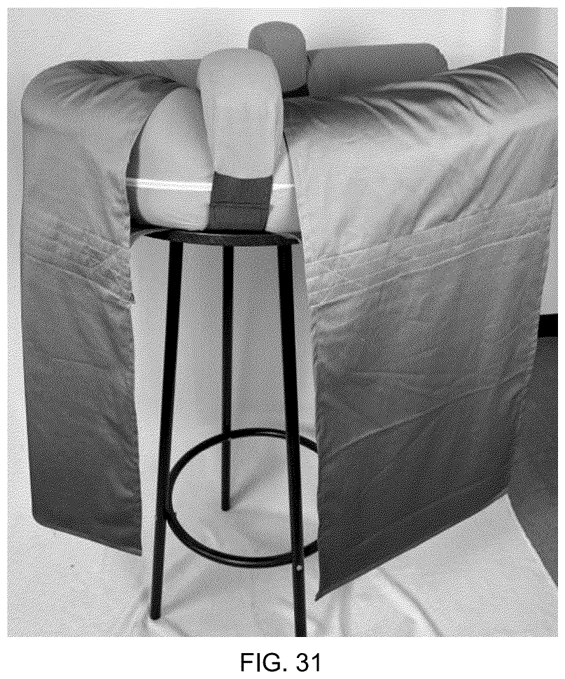

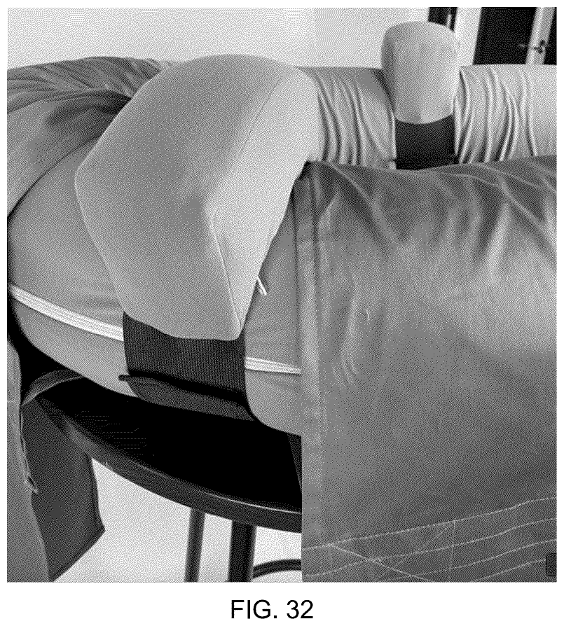

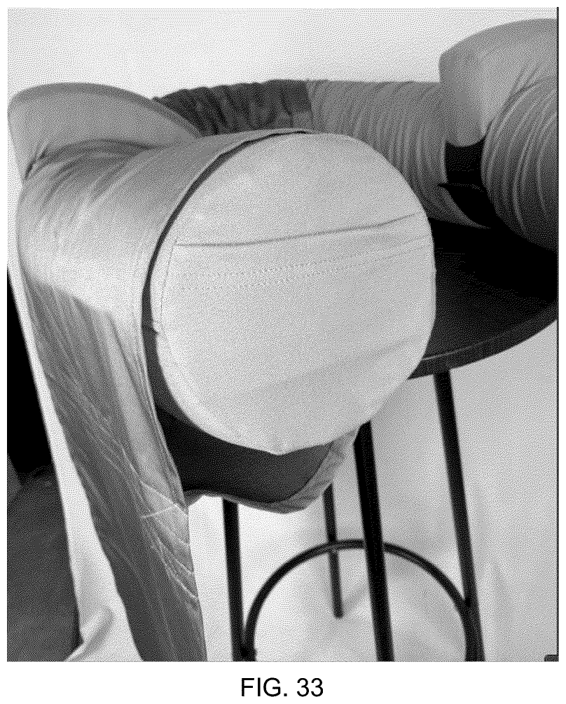

FIG. 29 is a perspective view of an embodiment of a prototype arm support apparatus that has been reduced to practice in accordance with one or more aspects and features of the invention. Additionally, FIG. 30 is a perspective view of the arm support apparatus of FIG. 29; FIG. 31 is another perspective view of the arm support apparatus of FIG. 29; FIG. 32 is a perspective, close-up view of a portion of the arm support apparatus of FIG. 29; and FIG. 33 is a perspective, close-up view of another portion of the arm support apparatus of FIG. 29.

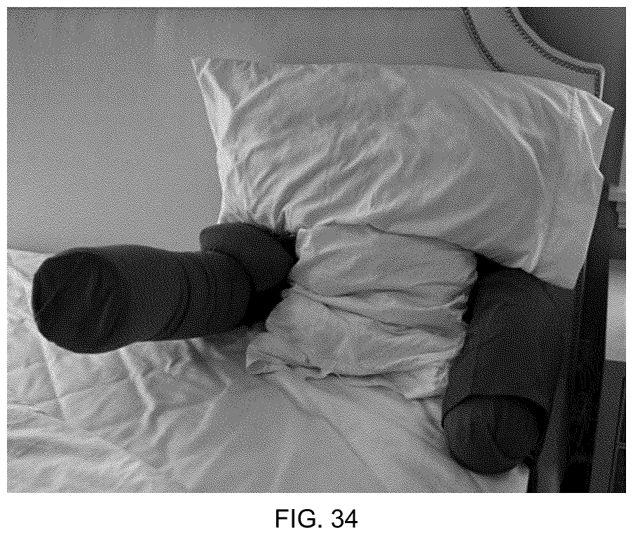

FIG. 34 is a perspective view of another prototype arm support apparatus installed on a bed and ready for use in accordance with one or more aspects and features of the invention. Pillows have been positioned at the bend in the tubular component for supporting a person's back in a sitting position. Preferably, the person is reclined between 30 to 90 degrees when in the sitting position for resting, including sleeping, and more preferably in a reclined position of about 35-45 degrees for comfort.

FIG. 35 is a perspective view of the arm support apparatus of FIG. 34 being used in accordance with one or more aspects and features of the invention. As shown in FIG. 34, the free end of the tubular component is configured to support an arm at an elevation above the person's thigh. The restriction component is shown limiting backward movement of the arm's elbow, too. FIG. 36 is another perspective view of the arm support apparatus of FIG. 34 in use in accordance with one or more aspects and features of the invention. As demonstrated in FIG. 36, an arm can be supported even when retained within a sling, brace, cast, or other immobilizing device.

Additional examples of use of arm support apparatus in accordance with one or more aspects and features of the invention are disclosed in the appendices and, in particular, in the videos of the computer program listing, incorporated herein by reference.

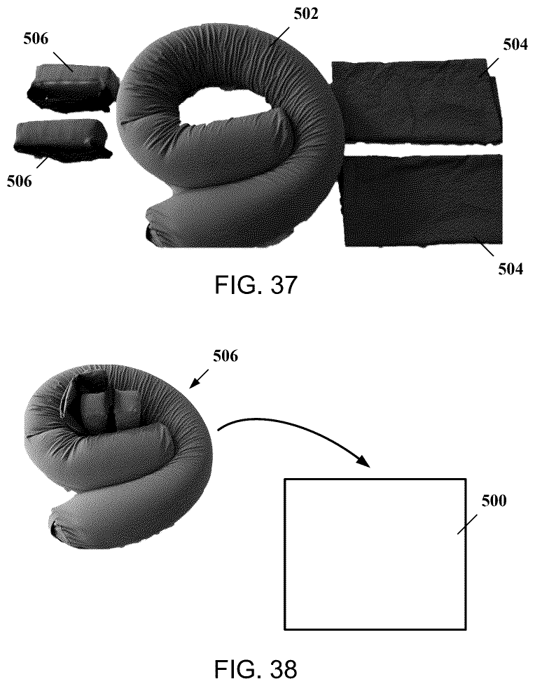

In commercializing the invention, the arm support apparatus preferably is sold and/or shipped in a container such as a box 500 and comprises five separate components for assembly by a user. FIG. 37 is a perspective view of the five separate components, which comprise a tubular component 502, two anchor components 504, and two restriction components 506. These five separate components preferably are bundled as shown at 506 with the tubular component 502 configured to wrap around and contain the other components 504,506, and these five components are disposed within the box 500 as illustrated in FIG. 38 for storage or shipment in accordance with one or more aspects and features of the invention. It is believed that the total weight of the box with these components would be about nine pounds and that the box dimensions would be 24″×24″×8″.

Based on the foregoing description, it will be readily understood by those persons skilled in the art that the invention has broad utility and application. Many embodiments and adaptations of the invention other than those specifically described herein, as well as many variations, modifications, and equivalent arrangements, will be apparent from or reasonably suggested by the invention and the foregoing descriptions thereof, without departing from the substance or scope of the invention.

For example, embodiments of the invention may be used in conjunction with a use on couch or sofa or a bed comprising other than a mattress and box spring.

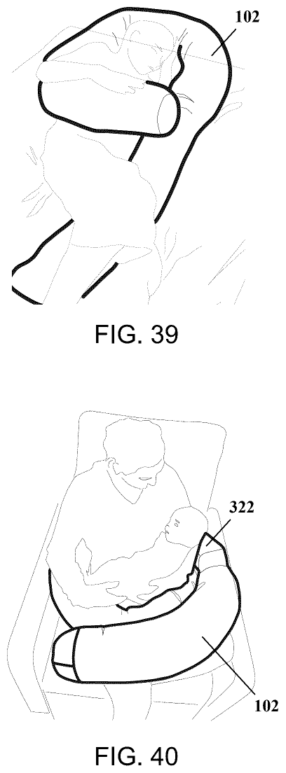

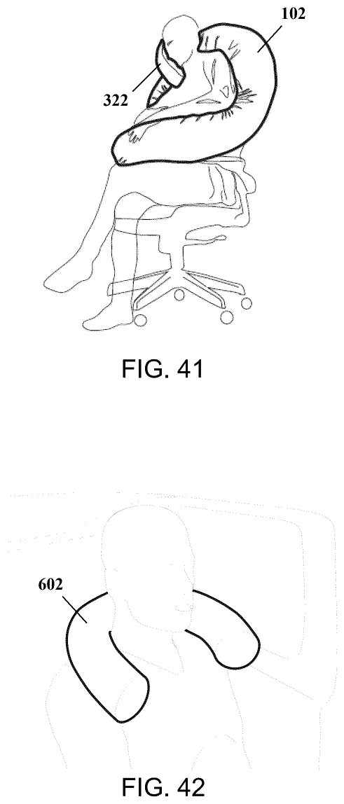

Additionally, one or more of the components of an arm support apparatus may be used in other contexts. In this regard, FIGS. 39-42 illustrate uses in such different contexts. Specifically, FIG. 39 is an illustration of a use of just a tubular component 102 for sleeping in accordance with one or more aspects and features of the invention; FIG. 40 is an illustration of a use of just a tubular component 102 and a restriction component 322 for caring for baby in accordance with one or more aspects and features of the invention; FIG. 41 is an illustration of a use of embodiments of a tubular component 102 and a restriction component 322 for sitting in an office chair in accordance with one or more aspects and features of the invention; and FIG. 42 is an illustration of a use of another embodiment of a tubular component 602 as a neck pillow when traveling such as in a car or on an airplane in accordance with one or more aspects and features of the invention.

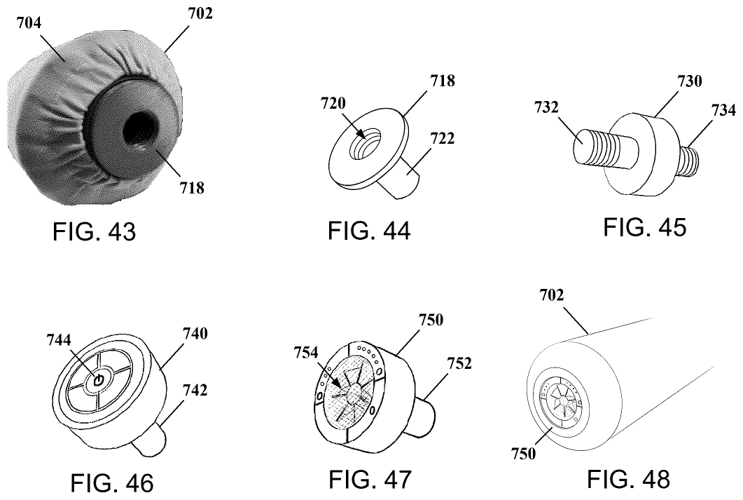

Accessories are also contemplated for use with an alternative embodiment of a tubular component in accordance with one or more aspects and features of the invention. In this regard, an end of such a tubular component 702 is shown in FIG. 43. An outer fabric covering 704 of the tubular component 702 does not cover the end of the tubular component 702 such that an end of the interior spine subcomponent is exposed and the interior passageway of the spine subcomponent is made accessible. As shown in FIG. 43, an end cap subcomponent 718 is inserted into the interior passageway of the end of the spine subcomponent to form the outermost end of the tubular component 702.

The end cap subcomponent 718 is shown by itself in FIG. 44. The end cap subcomponent 718 has a threaded opening 720 on one side and a plug portion 722 on the opposite side. The plug portion 722 fits in friction fit within the interior passageway of the spine subcomponent at the exposed end of the interior spine subcomponent. The threaded opening 720 of the end cap subcomponent 718 is configured to receive in threaded engagement a threaded plug portion 732 of a quick connect subcomponent 730, which is shown in FIG. 45. The quick connect subcomponent 730 has an opposite threaded plug portion 734 that is identical to and interchangeable with the threaded plug portion 732 such that either threaded plug portion may be screwed into the threaded opening 720 of any end cap subcomponent 718. Multiple tubular components 702 may thereby join together in end-to-end relation to form a longer, composite tubular member. Moreover, each such tubular component 702 may have a shorter length than, for example, the tubular component 102 of FIG. 1, whereby composite tubular components of different length may be constructed for accommodating different persons and different contexts of use. In this scenario, a tubular component 702 may be, for example 2 or 3 feet in longitudinal length. Indeed, a single tubular component 702 preferably is used as a neck pillow as represented in FIG. 42 but then joined with other tubular components 702 for use, for example, when sleeping (FIG. 39) or resting in a chair (FIG. 41), attending to a child (FIG. 40), or in supporting an arm in a resting position (FIGS. 35 and 36).

Preferably, subcomponents other than end cap subcomponent 718 have a plug portion for insertion in friction fit into the interior passageway of the spine subcomponent of the tubular component 702. For example, FIG. 46 is an orthogonal view of a wireless speaker subcomponent 740 for use with a tubular component of which tubular component 702 FIG. 43 is representative. The wireless speaker subcomponent 740 comprises a plug portion 742 for insertion in friction fit into the interior passageway of the spine subcomponent of the tubular component 702. The wireless speaker subcomponent 740 preferably connects to an electronic device for playing music, such as an iPhone or Android device using Bluetooth communications. The wireless speaker subcomponent 740 further comprises an internal, rechargeable power source and a power button 744 for turning on and off the wireless speaker subcomponent 740.

In another example, FIG. 47 is an orthogonal view of an air circulation subcomponent 750 having a fan for use with a tubular component of which tubular component 702 FIG. 43 is representative. The air circulation subcomponent 750 comprises a plug portion 752 for insertion in friction fit into the interior passageway of the spine subcomponent of the tubular component 702, as shown in FIG. 48. Specifically, FIG. 48 is a perspective view of the air circulation subcomponent 750 inserted at the end of the tubular components. The heating/cooling component 750 further comprises a fan 754 and an internal heating mechanism, cooling mechanism, or both. The fan 754 causes air to flow from exterior of the tubular component into and through the interior passageway of the spine subcomponent. The spine subcomponent is ventilated as is the foam subcomponent and the fabric cover, whereby airflow exits the internal passageway. This is shown in FIG. 51, discussed below.

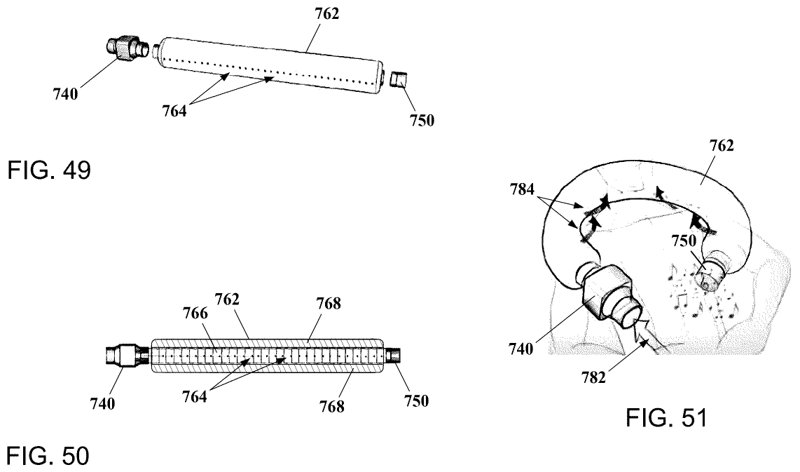

FIG. 49 is a perspective, exploded view of embodiments of a wireless speaker subcomponent 740 and the air circulation subcomponent 750 of a ventilated tubular component 762 in accordance with one or more aspects and features of the invention. The ventilated tubular component 762 comprises an array of ventilation passageways 764 forming one or more rows for airflow from the interior passageway of the spine subcomponent. FIG. 50 is an elevational view of the wireless speaker subcomponent 740 and the air circulation subcomponent 750 of the ventilated tubular component 762 of FIG. 50. A portion of the tubular component 762 is shown in cross-sectional view to reveal a ventilated spine subcomponent 766 surrounded by a foam subcomponent 768.

FIG. 51 is an illustration of a use of the air circulation subcomponent 740 and the wireless speaker component 750 of the tubular component 762 of FIG. 50. The tubular component 762 is wrapped around a persona's neck and is being used as a neck pillow while music plays from the wireless speaker subcomponent 740. Moreover, an air flows as indicated at 782 into and through the air circulation subcomponent 750, where it is preferably heated or cooled as desired, and then flows into and through the internal passageway of the spine subcomponent and through the ventilation passageways of the tubular component 762 and then out of the tubular component 762 as indicated at 784. Furthermore, the air circulation subcomponent 740 may instead comprise a fan without a heating and cooling mechanism, in which case ambient air is simply circulated through and out of the tubular member.

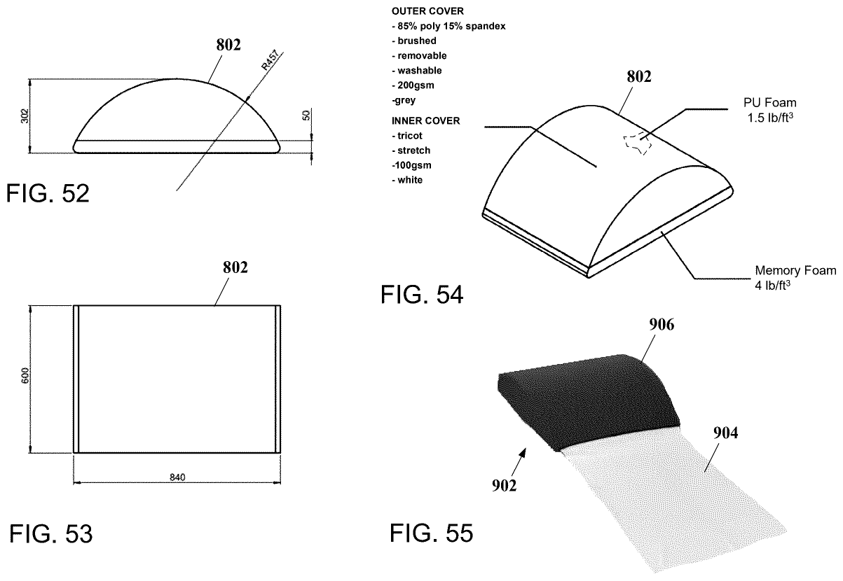

In addition to the foregoing, it is further contemplated that an optional foam component may be used with one or move embodiments for supporting one or more legs of a person as a leg rest or ottoman when sitting on a couch or bed. FIG. 52 is an elevational, orthogonal view of such an optional component 802 for use with an arm support apparatus in accordance with one or more aspects and features of the invention. FIG. 53 is a top plan, orthogonal view of the component 802; and FIG. 54 is another orthogonal view of the component 802. The support component 802 comprises a first foam subcomponent and a second foam subcomponent that form a composite cushion subcomponent 806.

FIG. 55 is a perspective view of an alternative optional component 902 similar to that of FIGS. 52-54. The alternative optional component 902 includes a sheet subcomponent 904 attached at a longitudinal end to a composite cushion subcomponent 906 and extending away to an opposite longitudinal end that is unattached to anything. Similar to an end portion 110 of an anchor component, the sheet subcomponent 906 is positioned so as to anchor the composite cushion component during use of the cushion subcomponent 906.

Accordingly, while the invention has been described herein in detail in relation to one or more preferred embodiments, it is to be understood that this disclosure is only illustrative and exemplary of the invention and is made merely for the purpose of providing a full and enabling disclosure of the invention. The foregoing disclosure is not intended to be construed to limit the invention or otherwise exclude any such other embodiments, adaptations, variations, modifications or equivalent arrangements, the invention being limited only by the claims appended hereto and the equivalents thereof.

What is claimed is:

1. An arm support apparatus, comprising a tubular component and one or more anchor components.

2. The arm support apparatus of claim 1, wherein an anchor component comprises a fabric sheet.

3. The arm support apparatus of claim 2, wherein the anchor component comprises hook-and-loop fasteners for securing an end edge portion of the fabric sheet to itself in overlapping manner so as to define a passageway for accommodating extension of the tubular component therethrough.

4. The arm support apparatus of claim 2, wherein the anchor component comprises an end edge portion of the fabric sheet that is sewn or otherwise permanently attached to itself so as to define a passageway for accommodating extension of the tubular component therethrough.

5. The arm support apparatus of claim 1, wherein the tubular component comprises a foam subcomponent and a spine subcomponent, both of which are enclosed within an outer fabric covering of the tubular component.

6. The arm support apparatus of claim 5, wherein the spine subcomponent is rigid but also manually bendable and enables the tubular component to be manually manipulated into and remain in different configurations.

7. The arm support apparatus of claim 6, wherein the tubular component is transitionable between a horseshoe configuration, a generally straight configuration, and a spiral configuration.

8. The arm support apparatus of claim 7, wherein each configuration of the tubular component is stable insofar as the tubular component remains in such configuration until a sufficient force is applied for manually manipulating the tubular component into a different configuration.

9. The arm support apparatus of claim 8, wherein such a force is greater than the force arising from the weight of an arm that is supported at an elevation by the tubular component.

10. The arm support apparatus of claim 1, wherein each anchor component comprises a fabric sheet that has a first end portion that is secured on a portion of the tubular component and that has an opposite end portion that freely drapes or hangs from the tubular component.

11. The arm support apparatus of claim 10, wherein the first end portion of the anchor component is doubled over on itself with an end edge portion being sewn or otherwise attached to itself at such that the tubular component is hemmed in by the first end portion.

12. The arm support apparatus of claim 10, wherein the first end portion of the anchor component surrounds the tubular component and forms a passageway through which the tubular component extends, thereby enabling the anchor component to be secured onto the tubular component without fixing the location at which it is secured and enabling the anchor component to be repositioned along the tubular component by sliding the first end portion along the tubular component.

13. The arm support apparatus of claim 1, wherein the arm support apparatus comprises a pocket at each of its opposite distal ends.

14. The arm support apparatus of claim 13, wherein the pocket is configured for holding a mobile phone or remote-control device for a television or other audiovisual equipment.

15. The arm support apparatus of claim 13, wherein the pocket is formed by overlapping fabric sections of the tubular component.

16. The arm support apparatus of claim 1, wherein the tubular component comprises an outer fabric covering, and a zipper fastening mechanism for providing access to an interior of the tubular component.

17. The arm support apparatus of claim 1, wherein the tubular component comprises a foam subcomponent.

18. The arm support apparatus of claim 17, wherein the foam subcomponent defines an interior passageway located along a longitudinal core thereof.

19. The arm support apparatus of claim 18, further comprising two end cap subcomponents that cap off the interior passageway of the foam subcomponent.

20. The arm support apparatus of claim 19, wherein the end cap subcomponents are adhered, welded, or otherwise attached to opposite distal ends of the foam subcomponent.

21. The arm support apparatus of claim 19, further comprising a spine subcomponent located within the interior passageway between the end cap subcomponents.

22. The arm support apparatus of claim 21, wherein the spine subcomponent comprises a modular tube or hose.

23. A method of making a foam subcomponent of a tubular component for an arm support apparatus, comprising transforming a sheet of foam into a tube and fixing the sheet of foam in the configuration of the tube.

24. The method of claim 23, wherein the foam is memory foam.

25. The method of claim 23, wherein the sheet of foam is transformed by rolling.

26. The method of claim 23, wherein the sheet of foam is fixed in the configuration of the tube by welding.

27. The method of claim 23, wherein the sheet of foam is fixed in the configuration of the tube using an adhesive.

28. An arm support apparatus, comprising a tubular component and one or more restriction components that are attachable to the tubular component and designed to limit the movement of the arm, and preferably the elbow, when used.

29. The arm support apparatus of claim 28, wherein the restriction component comprises a block and a strap.

30. The arm support apparatus of claim 29, wherein a distal end of the strap is permanently attached to an end of the block, and the other distal end of the strap is looped through a D-ring that is attached to another end of the block, whereby the strap extends back over itself to fasten proximate the first distal end.

31. The arm support apparatus of claim 30, further comprising hook-and-loop fasteners on the strap for detachably securing the second distal end to the strap proximate the first distal end after positioning of the strap around the tubular component for securing the restriction component to the tubular component.

32. The arm support apparatus of claim 28, wherein the block comprises an outer fabric covering, foam subcomponent enclosed within the outer fabric covering, and a zipper fastening mechanism providing access to the interior of the block and the enclosed foam subcomponent.

33. The arm support apparatus of claim 32, wherein the foam subcomponent comprises a memory foam and defines and gives shape to the block.

34. A method of providing an arm support apparatus, comprising disposing five separate components for assembly by a user in a container, the five separate components comprising a tubular component, two anchor components, and two restriction components.

35. The method of claim 34, wherein the five separate components are bundled with the tubular component configured to wrap around and contain the other components.

36. The method of claim 34, wherein the tubular component is in a spiral configuration.

37. A method of installing an arm support apparatus of any of the foregoing claims on a bed, comprising positioning a tubular component along one or more edges of the bed and tucking an end portion of each anchor component between a mattress and a box spring of the bed.

38. The method of claim 37, wherein the arm support apparatus is positioned at a corner of the bed.

39. The method of claim 37, wherein the arm support apparatus includes two anchor members each located on a different side of the bed proximate a corner of the bed, whereby the anchor components retain the arm support apparatus to the bed and keep portions of the tubular component extending through the passageway and between the anchor components from moving away from the edges of the bed during use.

40. The method of claim 37, wherein the arm support apparatus includes a “free” end portion that is bendable and transitionable relative to a person sitting on the bed at the corner of the bed so as to support an inside arm of the person during use of the arm support apparatus.

41. The method of claim 40, wherein the tubular component preferably is between about five feet and about ten feet in longitudinal length between its opposite distal ends, with the free end portion being between about two feet and about three feet, or more, in length.

42. The method of claim 40, wherein the free end portion extends about 25% to about 40%, or more, of the total longitudinal length of the tubular component.

43. The method of claim 40, wherein about 60% to about 75% of the tubular component is secured by the anchor components along one or more edges of the bed when installed, with the remaining length of the tubular component being free to extend from the edge and manipulated into a desired configuration for support of an arm.

44. The method of claim 37, further comprising wrapping the anchor component about a portion of the tubular component and securing the anchor component to itself in a detachable manner.

45. The method of claim 37, further comprising wrapping the anchor component about a portion of the tubular component and securing the anchor component to itself using hook-and-loop fasteners.

46. The method of claim 37, further comprising positioning one or more pillows at a bend in the tubular component for supporting a person's back in a sitting position for supporting a person at an incline of between 30 to 90 degrees when in the sitting position for resting, including sleeping.

47. The method of claim 46, wherein the incline is between 35 and 45 degrees for comfort.

48. The method of claim 37, further comprising supporting an arm at an elevation above a person's thigh using the free end of the tubular component.

49. The method of claim 37, further comprising restricting backward movement of the arm's elbow using a restriction component.

50. The method of claim 49, wherein the arm is further retained within a sling, brace, or other immobilizing device.

51. The arm support apparatus of any of claims claim 1-22 or 28-33, further comprising a support component for supporting one or more legs of a person as a leg rest or ottoman when sitting.

52. The arm support apparatus of claim 51, wherein the support component comprises a first foam subcomponent and a second foam subcomponent forming a composite cushion subcomponent.

53. The arm support apparatus of claim 52, wherein the support component further comprises a sheet subcomponent attached at a longitudinal end to the composite cushion subcomponent and extending away to an opposite longitudinal end that is unattached and is configured to be positioned so as to anchor the composite cushion component during use.

54. A method of using a tubular component as disclosed herein for sleeping.

55. A method of using a tubular component as disclosed herein for resting in an office chair.

56. A method of using a tubular component as disclosed herein for attending to a child.

57. A method of using a tubular component as disclosed herein as a neck pillow when traveling.

58. A tubular component comprising a foam subcomponent having an outer fabric covering and a spine subcomponent extending along a longitudinal axis of the foam subcomponent and defining an interior passageway, wherein the outer fabric covering does not cover a longitudinal end of the tubular component such that an end of the interior spine subcomponent is exposed and the interior passageway of the spine subcomponent is made accessible, and wherein an end cap subcomponent is inserted into the interior passageway of the end of the spine subcomponent.

59. The tubular component of claim 58, wherein the end cap subcomponent forms an outermost end of the tubular component.

60. The tubular component of claim 59, wherein the end cap subcomponent has a threaded opening on one side and a plug portion on the opposite side, the plug portion fitting in friction fit within the interior passageway of the spine subcomponent at the exposed end of the interior spine subcomponent.

61. The tubular component of claim 60, wherein the threaded opening of the end cap subcomponent is configured to receive in threaded engagement a threaded plug portion of another quick connect subcomponent, the quick connect subcomponent having an opposite threaded plug portion.

62. A composite tubular member comprising multiple tubular components join together in end-to-end relation, each of the multiple tubular components comprising a foam subcomponent having an outer fabric covering and a spine subcomponent extending along a longitudinal axis of the foam subcomponent and defining an interior passageway, wherein the outer fabric covering does not cover a longitudinal end of the tubular component such that an end of the interior spine subcomponent is exposed and the interior passageway of the spine subcomponent is made accessible.

63. A tubular component comprising a foam subcomponent having an outer fabric covering and a spine subcomponent extending along a longitudinal axis of the foam subcomponent and defining an interior passageway, wherein the outer fabric covering does not cover a longitudinal end of the tubular component such that an end of the interior spine subcomponent is exposed and the interior passageway of the spine subcomponent is made accessible, and wherein a wireless speaker subcomponent is inserted into the interior passageway of the end of the spine subcomponent, the wireless speaker subcomponent comprising a plug portion for insertion in friction fit into the interior passageway of the spine subcomponent of the tubular component.

64. The tubular component of claim 63, wherein the wireless speaker subcomponent is configured to connect to an electronic device for playing music.

65. The tubular component of claim 63, wherein the wireless speaker subcomponent further comprises an internal, rechargeable power source and a power button for turning on and off the wireless speaker subcomponent while inserted in the interior passageway of the spine subcomponent of the tubular component.

66. A tubular component comprising a foam subcomponent having an outer fabric covering and a spine subcomponent extending along a longitudinal axis of the foam subcomponent and defining an interior passageway, wherein the outer fabric covering does not cover a longitudinal end of the tubular component such that an end of the interior spine subcomponent is exposed and the interior passageway of the spine subcomponent is made accessible, and wherein an air circulation subcomponent comprising a fan is inserted into the interior passageway of the end of the spine subcomponent, the air circulation subcomponent comprising a plug portion for insertion in friction fit into the interior passageway of the spine subcomponent of the tubular component, the air circulation subcomponent being configured to cause air to flow from exterior of the tubular component into and through the interior passageway of the spine subcomponent.

67. The tubular component of claim 66, wherein the air circulation subcomponent further comprises an internal heating mechanism, an internal cooling mechanism, or both for heating/cooling air flowing through the air circulation subcomponent.

68. The tubular component of claim 66, wherein the spine subcomponent is ventilated.

69. The tubular component of claim 66, further comprising ventilation passageways extending through the fabric cover, the foam subcomponent, and the spine subcomponent for facilitating air flow from the spine subcomponent out of the tubular component.

70. The tubular component of claim 69, wherein the ventilation passageways form an array of one or more rows of exterior openings for airflow from the interior passageway of the spine subcomponent out of the tubular component.

71. The tubular component of claim 66, further comprising a wireless speaker subcomponent inserted into the interior passageway of the opposite longitudinal end of the spine subcomponent, the wireless speaker subcomponent comprising a plug portion for insertion in friction fit into the interior passageway of the spine subcomponent of the tubular component.

72. The tubular component of claim 71, wherein the wireless speaker subcomponent caps the end of the interior passageway of the spine subcomponent and airflow longitudinally therethrough.
